(19) United States
(12) Patent Application Publication
(10) Pub. No.: US 2021/0172947 A1
(43) Pub. Date: Jun. 10, 2021

(54) METHOD

(71) Applicants: Muhunthan THILLAI, Cambridge (GB); IMPERIAL COLLEGE OF SCIENCE, TECHNOLOGY AND MEDICINE, London (GB)

(72) Inventors: Muhunthan THILLAI, Cambridge (GB); Ajit LALVANI, London (GB); Robert PARKER, London (GB)

(21) Appl. No.: 16/768,151

(22) PCT Filed: Nov. 29, 2018
(86) PCT No.: PCT/EP2018/082992

§ 371 Date: May 29, 2020

(30) Foreign Application Priority Data
Nov. 29, 2017 (GB) 1719853.2

(51) Int. Cl. G01N 33/569; G01N 33/68

(52) U.S. Cl. CPC G01N 33/5695; G01N 33/6893; G01N 2333/35; G01N 2800/7095; G01N 2333/7153; G01N 2800/12

(57) ABSTRACT

The present invention relates to a method for the diagnosis of sarcoidosis. In particular, the present invention relates to a method for the differential diagnosis of sarcoidosis versus tuberculosis infection.

### FIELD OF INVENTION

The present invention relates to a method for the diagnosis of sarcoidosis. In particular, the present invention relates to a method for the differential diagnosis of sarcoidosis versus tuberculosis infection.

### BACKGROUND

Pulmonary sarcoidosis (SA) and pulmonary tuberculosis (TB) are chronic granulomatous diseases with highly similar symptoms and radiological pathology (27156614) that pose a diagnostic challenge to clinicians. TB is caused by infection with Mycobacterium tuberculosis (Mtb) and affects a third of the world's population (ISBN 978 92 4). SA has no known aetiology and is less common, with the highest annual incidence reported to affect 5-40/10000 people in northern Europe (9012596). A causative agent has not been identified in SA and patients do not respond to antimicrobial therapy, but respond favourably to immune suppression. Pulmonary SA is often misdiagnosed as TB (27156614, 3484866, 19200680) as they are epidemiologically associated and both can clinically present with hilar lymphadenopathy and symptoms of fever, malaise, fatigue, weight loss and reduced respiratory function. To make a strong clinical diagnosis of SA, clinicians need several levels of clinical and molecular evidence, often ruling out TB through microbiological testing. A diagnosis of SA supported by a negative tuberculin skin test (TST) or interferon-gamma release assay (IGRA), elevated serum angiotensin-converting enzyme (SACE), bilateral hilar lymphadenopathy and non-necrotizing granulomas at the site of disease. Granulomas form in the lung in both pulmonary SA and TB; composed of immune cells (predominantly fused macrophages and CD4+ T-cells), they result from a host-protective response which acts to contain pathogens or other foreign material. In active TB, Mtb infects lung resident macrophages and other cells (8419415) (22517424) which aggregate to form primary granulomas that are unable to control infection due to Mtb virulence factors which promote lipid uptake, caseation and cell necrosis, leading to dissemination of bacteria through the lung. In immunocompetent individuals, a (Th1) immune response limits disease by providing antigen specific CD4+ T-cells able to activate macrophages to control mycobacterial replication within granulomas. Granulomas in SA also result from an ongoing Th1 immune response, but they are almost always non-caseous, epitheloid and sterile (9110911). In SA, chronic Th1 immune responses persist and have been found to be organism specific, responding to antigens from Mtb (katG, soda, Ag85A and hsp(s)) (17088357, 22284237) and Propionibacterium acnes (22552860). DNA from both Mtb and P. acnes have been found in SA granulomas, and SA may be triggered by infection in susceptible individuals that carry the DRB1*1101 allele (Ser. No. 19/536,643), indicating that in some cases SA may be an unwelcome outcome of a sterilised Mtb infection, or that both diseases maybe present simultaneously (20298409, 21446224).

Specific signatures derived from the blood are highly attractive as non-invasive, rapid and affordable tests able to support diagnosis of disease (23940611, 21852540). Currently whole blood transcriptomic studies in TB and SA indicate highly similar profiles with high inter-study variability in any disease specific gene signatures limiting diagnostic value of this approach (23940611, 21852540). The distinct biochemical make up of granulomas in TB and SA can be revealed through Endobronchial ultrasound-guided transbronchial needle aspiration (EBUS-TBNA) and this has improved diagnosis in mediastinal disease. The biochemical profiles derived from these distinct granulomas are found in the sera (e.g. SACE) and indicate that serum proteomic profiles may be able to distinguish SA and TB (22815689, 23399022, 26270185).

However, currently no biomarkers are available that provide a highly sensitive and specific clinical test. Methods of diagnosis require an invasive biopsy and the histological identification of distinct cellular features, this procedure comes with attendant risks and costs. Sarcoidosis is unresponsive to Tuberculosis therapy, with effective treatment requiring suppression of the ongoing immune response with corticosteroids. Often Sarcoidosis remains undiagnosed until other diseases are excluded, and whilst some individuals may self-cure, for many the disease progresses leading to pulmonary lung fibrosis and permanent difficulty breathing. A rapid and affordable diagnostic test which could discriminate between these two conditions would dramatically improve time to diagnosis and treatment for individuals with either condition.

### SUMMARY OF INVENTION

In a first aspect, the present invention relates to a method of differentiating between sarcoidosis and tuberculosis infection in a subject, the method comprising: (a) measuring the level of a sarcoidosis-specific biomarker in a sample taken from the subject; (b) measuring the level of a tuberculosis-specific biomarker in the same sample; (c) calculating the ratio of the sarcoidosis-specific biomarker to the tuberculosis-specific biomarker; and (d) comparing the ratio of the sarcoidosis-specific biomarker to the tuberculosis-specific biomarker to one or more standard values, where a calculated ratio less than a standard value is indicative of sarcoidosis and a calculated ratio higher or equal to a standard value is indicative of tuberculosis infection.

Four markers have been identified as useful in the method of the present inventions: Fibrinogen alpha chain (FGA), Protein S100-A9 (S100A8/A9), Macrophage colony-stimulating factor 1 receptor (MCSF1R or CSFR1) and Inter-alpha-trypsin inhibitor heavy chain H1 (ITIH1). Preferably the sarcoidosis-specific biomarker is Colony stimulating factor 1 receptor (CSFR1) and the tuberculosis-specific biomarker is S100A8A9 (calprotectin). More preferably, when the sarcoidosis-specific biomarker is CSFR1 and the tuberculosis-specific biomarker is S100A8A9 (calprotectin), the standard value is 2.

The standard value or values to which the calculated ratio is compared may vary depending on various factors, including but not limited to the age, gender, race or geographical location of the subject. In certain preferred embodiments, the standard value is any number from 1.5 to 5.0, preferably 1.5, 2.0, 2.5, 3.0, 3.5, 4.0, 4.5 or 5.0. In a particularly preferred embodiment, the standard value is 2.0. In other preferred embodiments, the calculated ratio is compared to multiple standard values in order to provide a qualitative assessment of the severity of the infection or of the reliability of the diagnosis.

A variety of samples may be used in the method of the present invention, including but not limited to blood, plasma, serum, lymph, pleural fluid, sputum, saliva, urine, and cerebrospinal fluid. In a preferred embodiment, the sample is a blood sample. In another preferred embodiment, the sample is a serum sample.

Measurement of the level of the relevant biomarkers may be carried out by any suitable method known in the art. In a preferred embodiment, the measurement of the level of the relevant biomarkers is carried out using ELISA.

Preferably the subject is a mammal, more preferably a human.

In a second aspect, the present invention relates to a method of diagnosing sarcoidosis in a subject, the method comprising: (a) measuring the level of a sarcoidosis-specific biomarker in a sample taken from the subject; (b) measuring the level of a tuberculosis-specific biomarker in the same sample; (c) calculating the ratio of the sarcoidosis-specific biomarker to the tuberculosis-specific biomarker; and (d) comparing the ratio of the sarcoidosis-specific biomarker to the tuberculosis-specific biomarker to one or more standard values, where a calculated ratio less than a standard value is indicative of a positive sarcoidosis diagnosis.

In a third aspect, the present invention relates to a method of diagnosing sarcoidosis in a subject, the method comprising: (a) measuring the level of CSFR1 in a sample taken from the subject; and (d) comparing the level of CSFR1 to one or more standard values, where a CSFR1 level above a standard value is indicative of a positive sarcoidosis diagnosis.

In a fourth aspect, the present invention relates to a method of treating tuberculosis or sarcoidosis in a subject in need thereof, wherein the method comprises:
(a) measuring the level of a sarcoidosis-specific biomarker in a sample taken from the subject;
(b) measuring the level of a tuberculosis-specific biomarker in the same sample;
(c) calculating the ratio of the sarcoidosis-specific biomarker to the tuberculosis-specific biomarker;
(d) comparing the ratio of the sarcoidosis-specific biomarker to the tuberculosis-specific biomarker to one or more standard values, where a calculated ratio less than a standard value is indicative of sarcoidosis and a calculated ratio higher or equal to a standard value is indicative of tuberculosis infection; and
(e) where the calculated ratio is less than a standard value, administering a therapeutic agent for sarcoidosis; or
(f) where the calculated ratio is higher than or equal to a standard, administering a therapeutic agent for tuberculosis.

This aspect of the invention also extends to a therapeutic agents for sarcoidosis or tuberculosis for use in a method of treating sarcoidosis or tuberculosis, wherein the method comprises:
(a) measuring the level of a sarcoidosis-specific biomarker in a sample taken from the subject;
(b) measuring the level of a tuberculosis-specific biomarker in the same sample;
(c) calculating the ratio of the sarcoidosis-specific biomarker to the tuberculosis-specific biomarker;
(d) comparing the ratio of the sarcoidosis-specific biomarker to the tuberculosis-specific biomarker to one or more standard values, where a calculated ratio less than a standard value is indicative of sarcoidosis and a calculated ratio higher or equal to a standard value is indicative of tuberculosis infection; and
(e) where the calculated ratio is less than a standard value, administering the therapeutic agent for sarcoidosis; or
(f) where the calculated ratio is higher than or equal to a standard, administering the therapeutic agent for tuberculosis.

This aspect of the invention also extends to the use of therapeutic agents for sarcoidosis or tuberculosis in the manufacture of a medicament for the treatment of sarcoidosis or tuberculosis in a subject in need thereof by a method comprising:
(a) measuring the level of a sarcoidosis-specific biomarker in a sample taken from the subject;
(b) measuring the level of a tuberculosis-specific biomarker in the same sample;
(c) calculating the ratio of the sarcoidosis-specific biomarker to the tuberculosis-specific biomarker;
(d) comparing the ratio of the sarcoidosis-specific biomarker to the tuberculosis-specific biomarker to one or more standard values, where a calculated ratio less than a standard value is indicative of sarcoidosis and a calculated ratio higher or equal to a standard value is indicative of tuberculosis infection; and
(e) where the calculated ratio is less than a standard value, administering the therapeutic agent for sarcoidosis; or
(f) where the calculated ratio is higher than or equal to a standard, administering the therapeutic agent for tuberculosis.

In a fifth aspect, the present invention relates to a method of treating sarcoidosis in a subject in need thereof, the method comprising:
(a) measuring the level of a sarcoidosis-specific biomarker in a sample taken from the subject;
(b) measuring the level of a tuberculosis-specific biomarker in the same sample;
(c) calculating the ratio of the sarcoidosis-specific biomarker to the tuberculosis-specific biomarker;
(d) comparing the ratio of the sarcoidosis-specific biomarker to the tuberculosis-specific biomarker to one or more standard values, where a calculated ratio less than a standard value is indicative of a positive sarcoidosis diagnosis; and
(e) where a positive sarcoidosis diagnosis is determined in step (d), administering a therapeutic agent for sarcoidosis.

This aspect of the invention also extends to a therapeutic agent for sarcoidosis for use in a method of treating sarcoidosis in subject in need thereof, wherein the method comprises:
(a) measuring the level of a sarcoidosis-specific biomarker in a sample taken from the subject;
(b) measuring the level of a tuberculosis-specific biomarker in the same sample;
(c) calculating the ratio of the sarcoidosis-specific biomarker to the tuberculosis-specific biomarker;
(d) comparing the ratio of the sarcoidosis-specific biomarker to the tuberculosis-specific biomarker to one or more standard values, where a calculated ratio less than a standard value is indicative of a positive sarcoidosis diagnosis; and
(e) where a positive sarcoidosis diagnosis is determined in step (d), administering the therapeutic agent for sarcoidosis.

This aspect of the invention also extends to the use of a therapeutic agent for sarcoidosis in the manufacture of a medicament for the treatment of sarcoidosis in a subject in need thereof by a method comprising:
(a) measuring the level of a sarcoidosis-specific biomarker in a sample taken from the subject;
(b) measuring the level of a tuberculosis-specific biomarker in the same sample;
(c) calculating the ratio of the sarcoidosis-specific biomarker to the tuberculosis-specific biomarker;
(d) comparing the ratio of the sarcoidosis-specific biomarker to the tuberculosis-specific biomarker to one or more standard values, where a calculated ratio less than a standard value is indicative of a positive sarcoidosis diagnosis; and
(e) where a positive sarcoidosis diagnosis is determined in step (d), administering the therapeutic agent for sarcoidosis.

In a sixth aspect, the present invention relates to a method of treating sarcoidosis in a subject in need thereof, wherein the method comprises:
(a) measuring the level of CSFR1 in a sample taken from the subject;
(b) comparing the level of CSFR1 to one or more standard values, where a CSFR1 level above a standard value is indicative of a positive sarcoidosis diagnosis; and
(c) administering a therapeutic agent for sarcoidosis.

This aspect of the invention also extends to a therapeutic agent for sarcoidosis for use in a method of treating sarcoidosis in subject in need thereof, wherein the method comprises:
(a) measuring the level of CSFR1 in a sample taken from the subject;
(b) comparing the level of CSFR1 to one or more standard values, where a CSFR1 level above a standard value is indicative of a positive sarcoidosis diagnosis; and
(c) administering the therapeutic agent for sarcoidosis.

This aspect of the invention also extends to the use of a therapeutic agent for sarcoidosis in the manufacture of a medicament for the treatment of sarcoidosis in a subject in need thereof by a method comprising:
(a) measuring the level of CSFR1 in a sample taken from the subject;
(b) comparing the level of CSFR1 to one or more standard values, where a CSFR1 level above a standard value is indicative of a positive sarcoidosis diagnosis; and
(c) administering the therapeutic agent for sarcoidosis.

All preferred features of the second and subsequent aspects of the invention are as for the first aspect mutatis mutandis.

### DESCRIPTION OF FIGURES

The present invention will be further understood by reference to the following figures, in which:

FIG. 1. Proteomic Analysis of Sarcoidosis and Tuberculosis serum. (A) Venn diagram showing the number of overlapping serum proteins in patients with TB and SA which are significantly changed (Welsh's T-test, FDR<5%). (B) Scatter plot of 2-D functional enrichment analysis for significant (FDR<5%). Plot is of the average ratio compared to healthy controls for proteins in each functional group. (C) Scatter plot of the in-patient ratios (CSF1R÷S100A8/A9) calculated using mass spectrometry (MS) and ELISA. Spearman's correlation P-value is shown. (D) Receiver Operating Characteristic (ROC) curve comparing SA and TB using ELISA measurements for CSF1R, S100A8/A9 protein complex and the in-patient ration of CSF1R:S100A8/A9 complex. The true positive rate (Sensitivity) is plotted in function of the false positive rate (100-Specificity) for different cut-off points.

FIG. 2. TB serum protein signature is associated with blood neutrophils and platelets number in peripheral blood. (A) Heat map of a Spearman's rank correlation matrix analysis of the median expression of proteins in each functional category (y) with the number different cells in a patient's blood (x). (B-F) Plots of Spearman's correlation of ELISA measurements for proteins (y) derived from the TB proteomic signature against the NLR and total Lymphocytes in a patient's blood (x).

FIG. 3 TB serum protein signature is associated with necrosis. (A-B) Bar plot of dsDNA (A) and Nucleosomes (B) measured in patient blood. (C) Receiver Operating Characteristic (ROC) curve comparing SA and TB using dsDNA and Nucleosome measurements. The true positive rate (Sensitivity) is plotted in function of the false positive rate (100-Specificity) for different cut-off points. (D) Bar plots of ELISA data for proteins derived from the TB signature for Healthy individuals (n=8), and individuals with TB and SA (n=41), paired with a scatter plot and Spearman's correlation analysis for four proteins from the TB proteomic signature with total Nucleosomes in each patient.

FIG. 4. Classification accuracy of in-patient ratio of S100A8/A9 and CSF1R in a diverse disease cohort. (A) Dot plot of the in-patient ratio for CSF1R:S100A8/A9 complex in validation cohort (n=88), sub-stratified dependent on time since diagnosis for SA. (B) Receiver Operating Characteristic (ROC) curve comparing SA and TB using the in-patient ratio of for CSF1R:S100A8/A9 complex in validation cohort (n=88), sub-stratified dependent on time since diagnosis for SA. The true positive rate (Sensitivity) is plotted in function of the false positive rate (100-Specificity) for different cut-off points.

FIG. 5. Diagram showing the patient numbers used in the three experiments performed, where the same patient sample is used in the same experiment the overlap is indicated.

FIG. 6. (A) Dot plots of median relative protein expression for all proteins in serum proteome for each Gene ontology (GO) category identified for in each disease group (HC, TB, SA). (B) Scatter plot and correlation analysis for median relative protein expression for each Gene ontology (GO) with underlying blood cell counts. (C) Line plot of the normalised expression of mRNA in different immune cell types for the 5 proteins identified by proteomic analysis (green=SA v HC) and an equal number of proteins chosen at random (black).

FIG. 7. (A) PCA analysis of proteomic data. (B) Box and whisker plots for the 4 best classifiers (S100A8/A9, CSF1R, ITIH1 and FGA) selected by random forest algorithm. (C) Receiver Operating Characteristic (ROC) curve comparing SA and TB using the four proteins selected by random forest machine learning.

FIG. 8. Scatter plot and Spearman's correlation analysis for proteins (S100A8/A9, CSF1R, ITIH1 and FGA) detected by both mass spectrometry and ELISA.

FIG. 9. (A) Bar plot of ELISA data for the in-patient ratio of CSF1R:S100A8/A9 protein complex for SA and TB using discovery cohort. (B) Receiver Operating Characteristic (ROC) curve comparing SA and TB using the in-patient ratio for CSF1R:S100A8 or A9 as determined from LC-MS data.

FIG. 10. (A) Individual dot and scatter plots of the in-patient ratio for CSF1R and S100A8/A9 complex in SA validation cohort (n=88), sub-stratified dependent on time since diagnosis for SA.

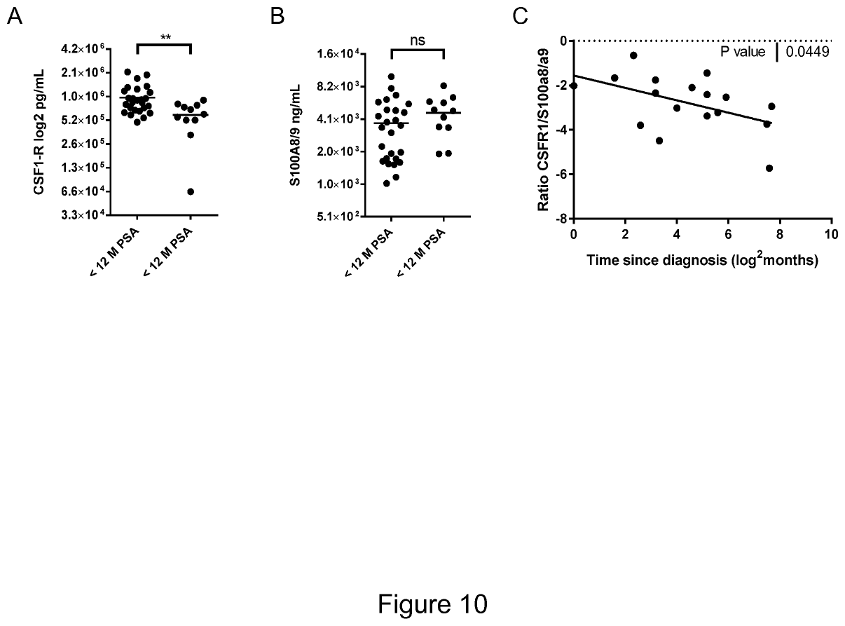

FIG. 11. A comparison of (a) a randomly chosen marker pair; (b) the preferred CSFR1/S100A8/A9 marker pair; and (c) a pair of markers composed of a known sarcoidosis marker (Chitotriosidase-1) and a known tuberculosis marker (Matrix metalloproteinase-9).

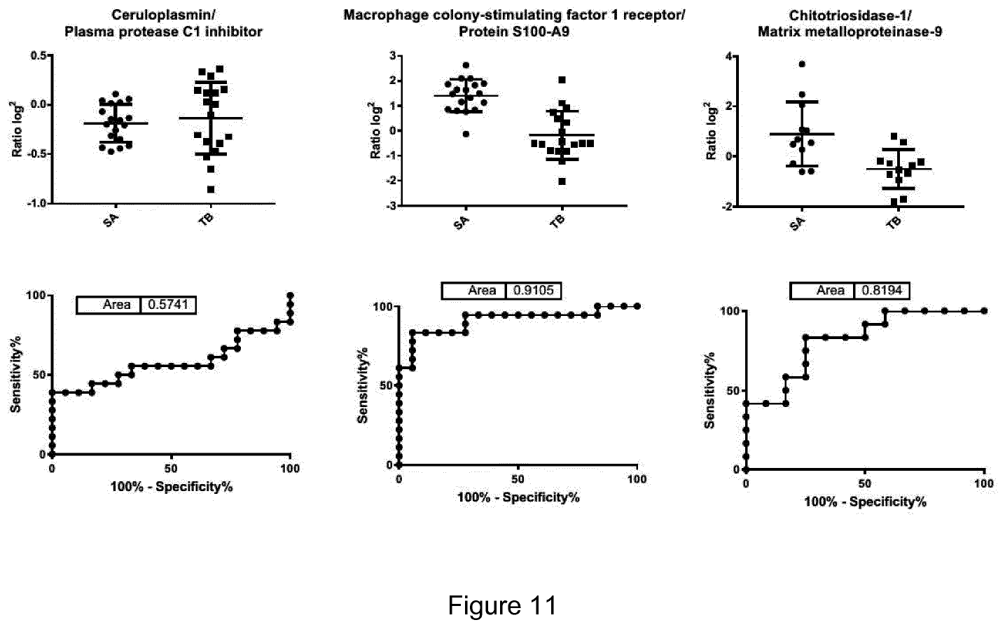

### DETAILED DESCRIPTION

Sarcoidosis is a chronic inflammatory disease that often results in a skin rash, shortness of breath and a persistent cough. It is not clear what the cause of sarcoidosis is, but it does require that certain susceptible individuals encounter antigens potentially from infectious organisms; however no single source of these antigens has been identified. A major clinical problem in hospitals is diagnosis of sarcoidosis as the disease shares symptomatic, radiological and immune-pathological features with the more common disease Tuberculosis that results from infection by Mycobacterium tuberculosis (Mtb). As Sarcoidosis is unresponsive to Tuberculosis therapy, a rapid and affordable diagnostic test which could discriminate between these two conditions would dramatically improve time to diagnosis and treatment for individuals with either condition.

Using high-resolution mass spectrometry, the present inventors have quantitatively assessed serum profiles of people (n=35) suffering from pulmonary forms of both diseases. 427 proteins were quantitated, of which 5 (CSF1R, CHIT1, LYZ1, APOE, ICAM1) were statistically significant in SA and 122 in TB disease. The in-patient ratio of two disease specific proteins (SA-CSF1R; TB-S100A8/A9) were explored by ELISA in a validation cohort (n=88) and provided high diagnostic accuracy (AUC=0.93). The inventors have therefore demonstrated that the serum proteome reflects the necrotic status often observed only through biopsy in SA and TB and can provide diagnostic value in these clinically similar pulmonary diseases.

The present invention therefore provides a platform for the differential diagnosis of sarcoidosis versus tuberculosis using markers identified via proteomic screening. In particular, CSFR1 is identified as a marker for sarcoidosis. CSFR1 has not previously been reported as a serum marker for sarcoidosis.

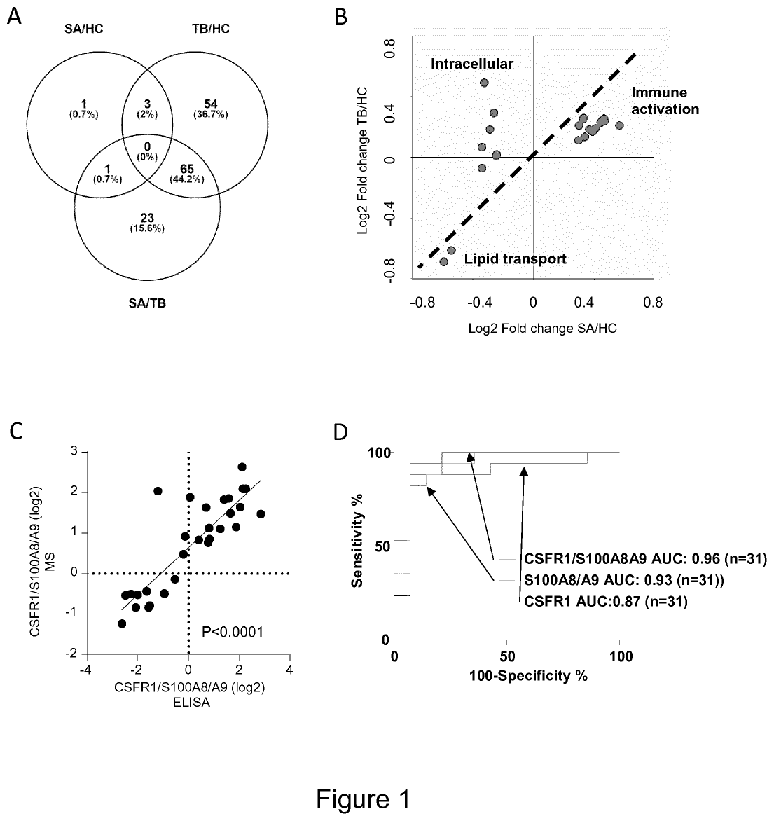

In further aspects, pairs of markers are identified that can be used to distinguish between sarcoidosis and tuberculosis. The present inventors have identified a pair of markers (CSFR1 and S100A8/A9) that allow for the differentiation of sarcoidosis and tuberculosis. These markers show improved differentiation when compared to a randomly chosen pair of markers (Ceruloplasmin/Plasma protease C1 inhibitor) and, significantly, the CSFR1/S100A8/A9 marker pair shows better differentiation when compared to a pair of markers composed of a known sarcoidosis marker (Chitotriosidase-1) and a known tuberculosis marker (Matrix metalloproteinase-9) (see FIG. 1).

The skilled person is aware of multiple methods to measure the level of a given marker in a sample and the choice of method depends on multiple factors, as will be understood by those of skill in the art. One particularly preferred method is the use of enzyme-linked immunosorbent assay (ELISA).

Typically, the subject is a mammal. In a preferred embodiment, the subject is a human. The sample may be any fluid or tissue sample from the subject, including but not limited to blood, plasma, serum, lymph, pleural fluid, sputum, saliva, urine, and cerebrospinal fluid. In a preferred embodiment, the sample is a blood sample.

### Examples

The invention will also be further described by way of reference to the following Examples which are present for the purposes of illustration only and are not to be construed as being limiting on the invention.

### Methods

### Sample Cohorts

Study participants were prospectively enrolled between 1 Sep. 2008 and 5 Jul. 2013 during routine National Health Service (NHS) screening for ATB or LTBI at one of the following NHS trusts in the United Kingdom: Imperial College Healthcare, Frimley Health, Bart's Healthcare, London Northwest. All participants were recruited under National Research Ethics Service (NRES) approval (07/H0712/85 and 11/H0722/8), provided informed consent after the nature and possible consequences of the studies were explained, and were aged >18 years. Individuals with known HIV were excluded from the study. SA and TB patients all had active pulmonary disease. TB samples were taken from microbiological culture confirmed cases or those diagnosed with TB based upon radiological features suggestive of TB (Ser. No. 18/316,751). SA patients were chosen based upon a clinical diagnosis defined by the American Thoracic Society guidelines (Ser. No. 10/430,755). Most SA patients had a biopsy and histological identification of non-necrotic granulomas, and were culture negative for Mtb when tested. No diagnostic screening for latent tuberculosis or SA was carried out for healthy controls but they all denied having any respiratory disease or other significant co-morbidity at the time of sample collection.

### Sample Processing

Blood was collected into a BD serum tube and allowed to clot for 60 mins before centrifuging at 1000 g for 10 mins at room temperature (RT) to remove cell debris and clots. All samples were stored at −80° C. within 90 mins of collection. At this point 54 serums matched for ethnicity, gender and age, 18 Sarcoidosis (PSA), 18 Tuberculosis (PTB) and 18 healthy control (HC)) were assigned to a blocked 10-plex balanced experimental design. Samples were distributed equally by disease and label into 3 blocks and randomly assigned a processing order using a random number table. Next, protein concentration was determined by Pierce BCA assay (Thermo Scientific, Waltham, Mass., USA.) and 600 ug of serum was immunodepleted of the top 12 abundant proteins using the Pierce Top 12 Abundant Protein Depletion Spin Columns (Thermo Scientific) according to the instructions. Depleted serum ˜800 ul was concentrated using a 3 Kda NMLW cut-off spin filter (Millipore) and protein precipitated by Chloroform methanol. Protein pellets were solubilised in 1% sodium deoxycholate, 100 mM Ammounium bicarbonate. 10 pg of protein was reduced with 10 mM DTT 15 minutes at 60° C. followed by alkylation with 20 mM Iodoacetamide for 15 minutes at room temperature in the dark. Trypsin (Promega, Madison, Wis., USA) was added at a 1:50 (enyzyme:protein) ratio and digestion carried out at 37° C. overnight. Peptide digests were purified using the 018 STop And Go Extraction (STAGE) tips and eluted peptides were dried and labelled with 9 labels from the TMT10plex Mass Tag Labelling Kit as described in instruction with minor modifications. Peptides were dissolved in 25 ul of 100 mM TEAB and 10 μL of each label in acetonitrile and incubated for 60 min at room temperature before it was quenched with 2.5 μL of 0.5 M Hydroxyamine and combined. Samples diluted to a final acetonitrile concentration of 3% acidified to 0.1% (v/v) trifluoroacetic acid purified again by the 018 STAGE tips and resolved into 6 fractions using SAX STAGE tips exactly as described in (Ser. No. 19/848,406). Each fraction was dried completely and dissolved in 2% (w/v) acetonitrile, 0.1% (v/v) formic acid prior to LC-MS.

### Mass Spectrometry, Protein Identification and Quantification

Samples were analysed using an EASY-nLC 1000 Liquid chromatography system coupled to a Q-Exactive mass spectrometer. The separation column and emitter was an EASY-Spray column, 50 cm×75 μm ID, PepMap C18, 2 μm particles, 100 Å pore size. Buffer A was 2% Acetonitrile, 0.1% formic acid and buffer B 100% (v/v) acetonitrile, 0.1% (v/v) formic acid. A gradient from 5% to 40% acetonitrile over 120 minutes was used to elute peptides for ionization by electrospray ionisation (ESI) and data dependent MS/MS acquisition consisting of 1 full MS1 (R=70K) scan acquisition from 350-1500m/z, and 10 HCD type MS2 scans (R=35K). MS/MS charge targets were limited to 1 E⁵ and isolation window set to 1.5 m/z, monoisotopic precursor selection, charge state screening and dynamic exclusion were enabled, charge states of +1, >4 and unassigned charge states were not subjected to MS2 fragmentation. Raw mass spectra were identified and quantified using Maxquant 1.5.15 using a 1% peptide and protein FDR. Searches were conducted against the uniprot SwissProt database downloaded on 06/06/14. The database was supplemented with common contaminant proteins introduced during proteomic experiments. Searches were specified as tryptic with 1 missed cleavage, 7 ppm precursor ion mass tolerance, 0.05 Da fragment ion mass tolerance, fixed modifications of carbamidomethylation (C), and variable modification of oxidation (M), acetylation (N-term, Protein). Reporter ion intensities for MS/MS scans were filtered to ensure <75% precursor isolation purity, summed and assigned to proteins based upon unique matches and parsimony as described previously. For protein quantitation the sum of all peptides reported intensities were pre-processed by log² transformation, checked for normality and z-score transformed to normalise between batches. To identify differentially expressed proteins, ANOVA and Welch test were calculated between all disease groups, P values were adjusted for the effect of multiple hypothesis testing using the FDR (<0.1) method.

ELISA Determination of Nucleosomes and dsDNA in Serum

Serum samples were defrosted on ice a maximum of two times, and the concentration of CSF1R, S100A8/A9, DEFA1, MPO and RBP4 was measured using commercial kits from R and D technologies. Samples were diluted with sample diluents specified in instructions, measured in duplicate, background corrected (450-520 nm) and concentration determined on a standard curve, the range of standard deviation for each test was (CSF1R, S100A8/A9, DEFA1, MPO and RBP4). Nucleosomes were measured in Serum diluted 1:4 using the Cell Death ELISA (Roche) as described in manufacturing instructions, and reported as a % of the positive control. dsDNA was measured directly in diluted serum using the Quant-iT™ PicoGreen dsDNA Assay Kit (Thermo Scientific). Data was not always normally distributed and statistical significance was assigned using the Mann-Whitley U-test where *P<0.05, **P<0.01, ***P<0.001 and ****P<0.0001, the Spearman's rank correlation and linear regression; all calculations were performed in Prism Graphpad V6 (Graphpad Software Inc, La Jolla, Calif., USA).

### In-Patient Ratio

Sera were diluted 1:600 in sample diluent and raw absorbance for CSF1R and S100A8/A9 ELISA were interpolated onto standard curves using purified proteins, but with identical numerical range to normalise data. The ratio was calculated by the following formula log² (CSF1R±S100A8/A9). The specificity, sensitivity and 95% confidence intervals were calculated using Prism Graph Pad.

### Results

TABLE 1
Clinical Characteristics of Pulmonary Sarcoidosis and
Tuberculosis in Discovery cohort

| Clinical Characteristic | SA (N = 17) | TB (N = 18) |
|---|---|---|
| Age, (mean years, SD) | 46 ± 14 | 41 ± 13 |
| Sex (% female) | 41% | 35% |
| Ethnic group | 15/2/1 | 16/2/1 |
| (white/black/Asian) | | |
| Diagnosis | Biopsy (n = 13) | Culture (n = 16) |
| | Clinical (n = 4) | Clinical (n = 2) |
| CT Stage | 1(n = 5) | |
| | 2(n = 8) | |
| | 3(n = 1) | |
| | 4(n = 1) | |
| | Unknown | |
| Time Since 1st | <12 month (n = 15) | <12 month (n = 18) |
| Diagnosis | >12 months (n = 2) | >12 months (n = 0) |
| Site of Disease | | |
| Lung (any) | n = 11 (64%) | n = 18 (100%) |
| Intra-thoracic Lymph | n = 3 (18%) | n = 0 |
| node (only) | | |
| Lung & Intra-thoracic | n = 8 (53%) | n = 0 |
| Lymph node (any) | | |
| Extra thoracic site (any) | n = 6 (35%) | n = 0 |

### Proteomic Analysis of Sarcoidosis and Tuberculosis Serum

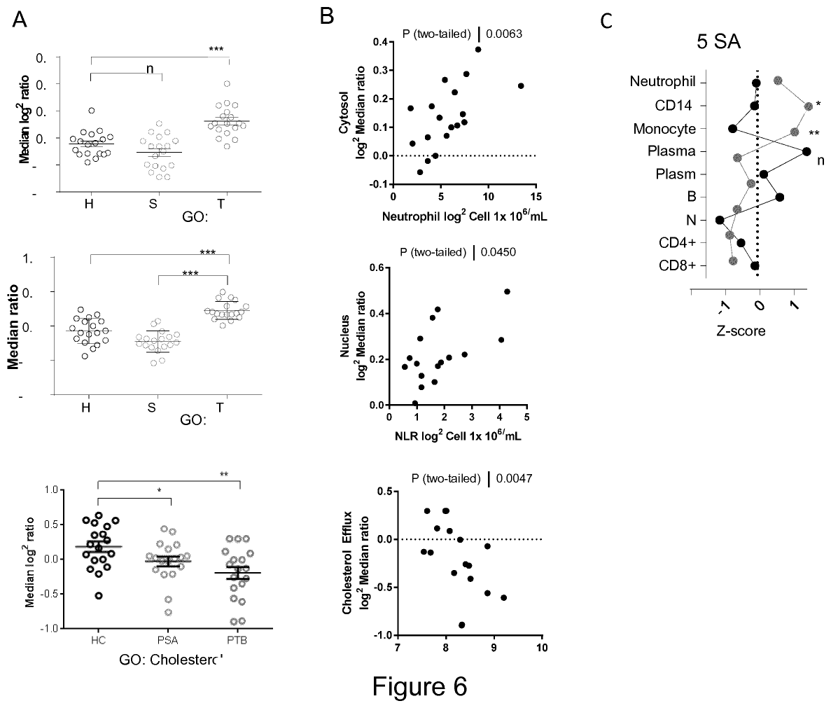

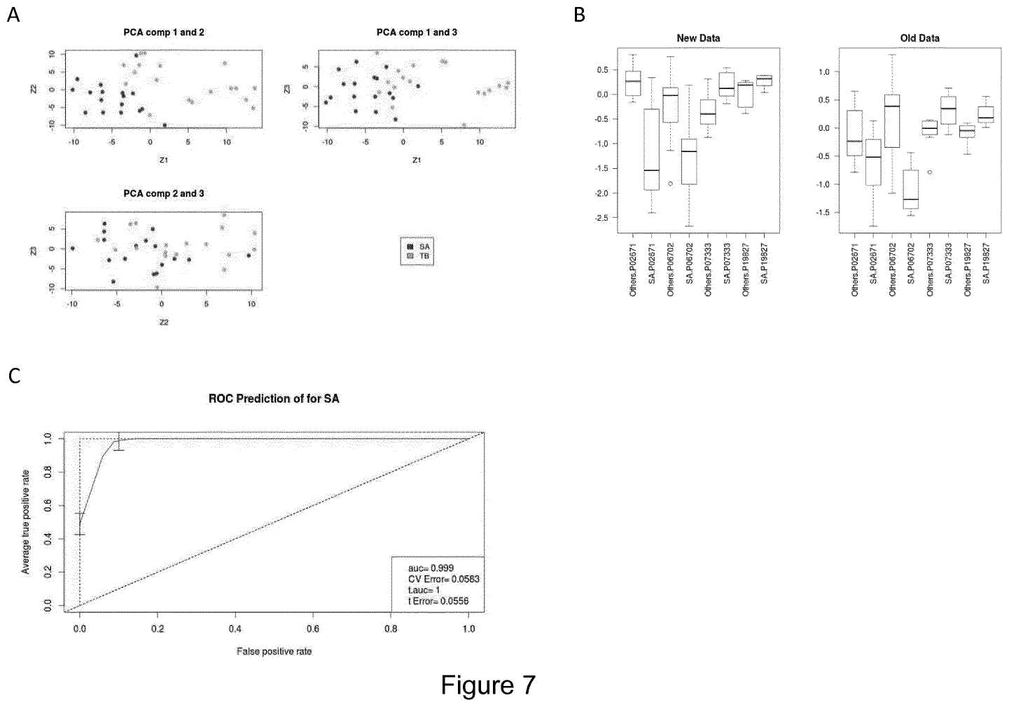

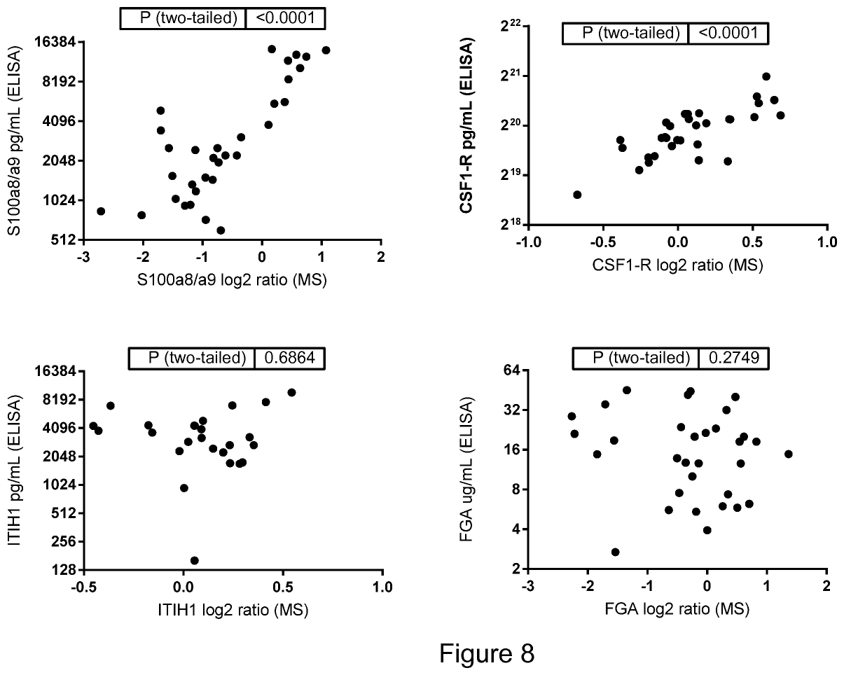

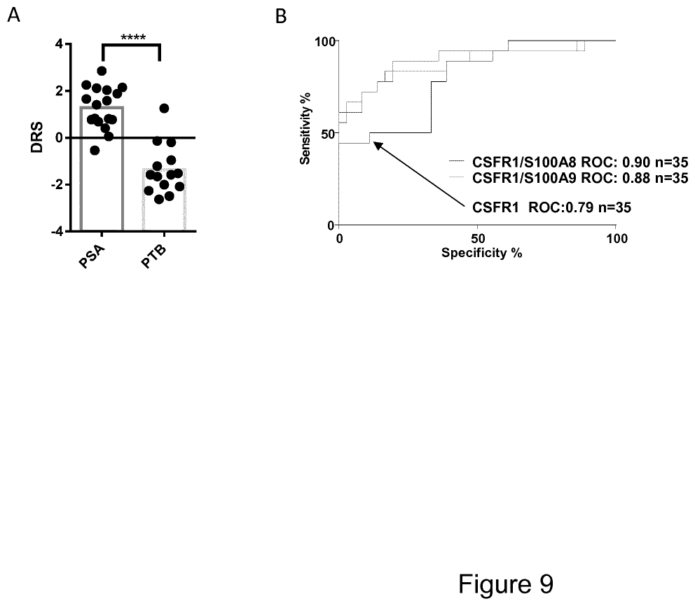

Using high-resolution mass spectrometry we generated a relative quantitative value for 427 proteins (FDR<0.05%) for a matched (age, ethnicity, gender) cohort of 35 patients with pulmonary SA or TB and 18 healthy people (HC). Disease specific changes (HC v TB, HC v SA, SA v TB, T-test, FDR<5%) show that TB affects the abundance of more proteins (n=122, 28.6% of total serum) than SA (n=5, 1.3% of total serum proteome) and that the protein CSF1R was both significantly different between SA v TB and SA v HC (FIG. 1 A). Functional gene-set enrichment using the Log^{e} fold change for TB and SA compared to healthy controls (23176165) identified several significant GO categories grouped as ‘immune activation’ and ‘lipid transport’ positively and negatively correlated for both diseases respectively (FIG. 1 B). Intracellular categories of cytoskeleton, cytosol and nucleus were specifically enriched in the serum of TB patients (FIG. 1 B, FIG. 6 A). No specific GO category was enriched for the 5 proteins identified in SA sera compered to HC. However a literature search for each protein revealed a shared role in macrophage biology and using published gene expression data we found the expression of each protein to be high in monocytes or CD14+ immune cell types (FIG. 6 C). To determine a useful diagnostic signature from our data samples were split into training (n=18) and test cohorts (n=17) based upon how the data was acquired. PCA analysis confirmed that the serum proteome of TB and SA were highly differentiated (FIG. 7 A) and machine learning (random forest) selected 4 protein classifiers (Fibrinogen alpha chain (FGA), Protein S100-A9 (S100A8/A9), Macrophage colony-stimulating factor 1 receptor (MCSF1R) and Inter-alpha-trypsin inhibitor heavy chain H1 (ITIH1) that could classify TB and SA with high accuracy (ROC AUC of 0.99) (FIGS. 7 B and C). We tested these proteins by ELISA and two S100A8/A9 complex and Macrophage colony-stimulating factor 1 receptor correlated well between LC-MS and ELISA platforms making them amenable to development of an ELISA based diagnostic test (FIG. 8). Using the normalised in-patient ratio of CSF1R and S100A8/A9 a simple diagnostic test was developed (FIG. 9). This test performed well, it correlated with MS data and provided a ROC AUC of 0.96 using ELISA to differentiate SA and TB (FIGS. 1 C and D, FIG. 9).

### TB Serum Proteomic Signature is Dependent Upon Patient Blood Cell Profile and Necrosis

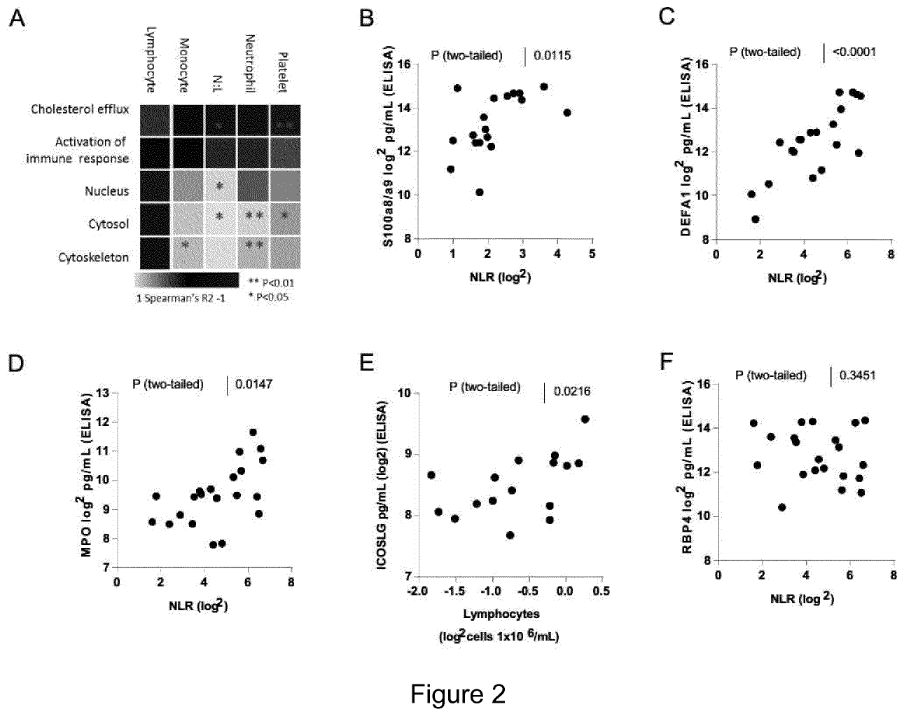

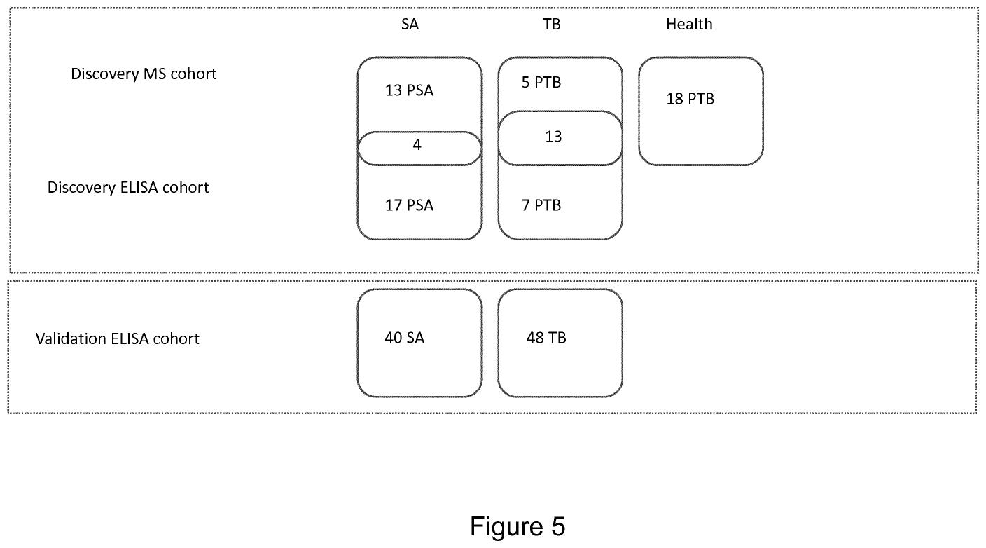

For 15/18 patients with TB the full blood cell count was available and a correlation analysis using the median protein ratio for GO categories revealed significant (Spearman's rank, P<0.05) positive correlations between total blood neutrophils and proteins from the intracellular categories of ‘Cytosol’ and ‘Cytoskeletal’ (FIG. 2 A, FIG. 6). Total platelet counts and Neutrophil lymphocyte ratio (NLR) negatively correlated with ‘Cholesterol efflux’ part of the Lipid transport group down regulated in TB. The correlation of individual proteins with blood immunology was investigated by ELISA for 5 TB protein biomarkers identified in our mass spectrometric screen. Using an extended ELISA discovery cohort consisting of 41 patients with TB or SA and 10 healthy individuals (FIG. 5) positive correlations for S100A8/A9 heterodimer (S100A8/A9) (P=0.0075), Myeloperoxidase (MPO) (P=0.0021), Defensin A3 (DEFA1) (P=0.0019) were detected with NLR. Inducible T-cell costimulator ligand (ICOSLG) (P=0.021) positively correlated with total blood lymphocyte counts, and there was no relationship between the liver protein Retinol binding protein 4 (RBP4) (P=0.35) and any blood immunological cell count (FIG. 2 A-F).

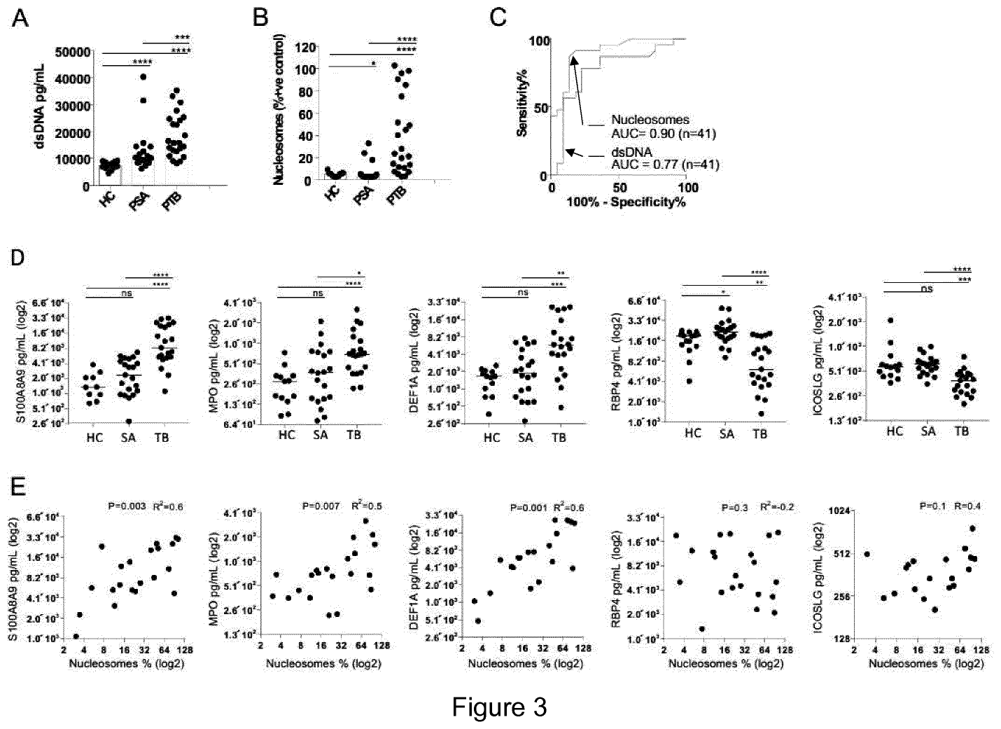

Because the proteomic screen indicated increased levels of intracellular proteins are a specific characteristic of TB sera we next determined cellular necrosis by measuring serum dsDNA and nucleosomes (FIG. 3 A, B). Serum dsDNA and nucleosomes increased in both TB and SA compared to healthy volunteers, the largest increase was observed for TB that was significantly more than SA (P=0.0016) (FIG. 3 A). Nucleosomes and dsDNA were also significantly different between TB and SA with nucleosome providing the highest diagnostic accuracy with a P<0.001 and an AUC of 0.90 (FIGS. 2 B and C). The ELISA for TB the biomarkers S100A8/A9, DEFA3, MPO, RBP4 and ICOSLG confirmed our LC-MS data and positive correlations with Nucleosomes in TB for S100A8/A9, DEFA3, MPO but not RBP4 or ICOSLG were detected (FIGS. 3 D and E).

Classification Accuracy of in-Patient Ratio of S100A8/A9 and CSF1R in a Diverse Disease Cohort

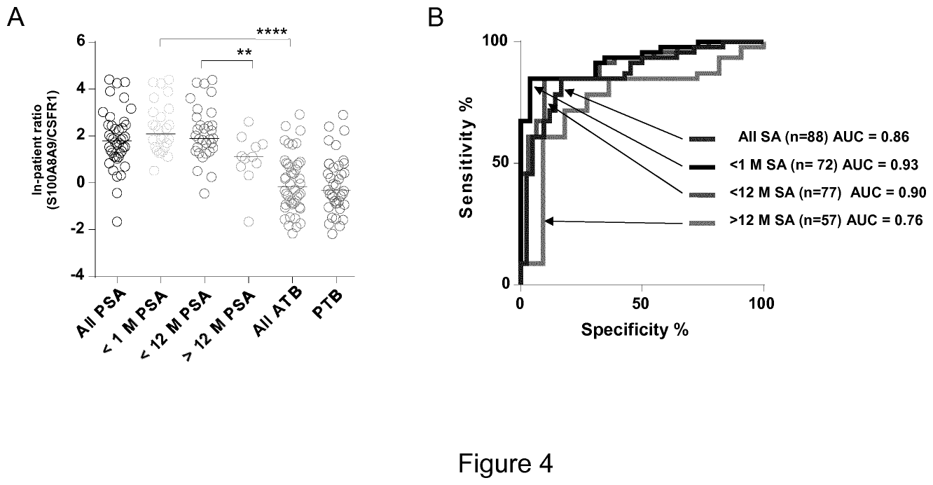

In order to investigate the diagnostic potential of S100A8/A9 and CSF1R to discriminate between TB and SA a new set of untreated SA and TB patients from the same cohort (n=88) was compiled to better reflect the full spectrum of both diseases including extra-pulmonary disease. Using the in-patient ratio a ˜2 fold cut-off was selected for disease classification and the accuracy and confidence intervals are reported in Table 4. Area under the curve analysis demonstrates that the test performs with similar accuracy for newly diagnosed SA (n=26) compared to TB (n=46) (ROC AUC=0.93, CI=0.87 to 0.98) (Table 4, FIGS. 4 A and B). Time since diagnosis for SA has the greatest effect on classification performance with a 1st diagnosis for SA greater than 12 month reducing specificity from 90% to 54%, this change is driven by a drop in the level of CSF1R in the serum of chronically diseased SA patients (FIG. 4. A, FIG. 8).

TABLE 3
Clinical Characteristics of Pulmonary Sarcoidosis and
Tuberculosis validation cohort

| | SA | TB |
|---|---|---|
| Clinical Characteristic | (N = 40) | (N = 48) |
| Age, (mean years, SD) | 45 ± 11 | 30 ± 14 |
| Sex (% female) | 53 | 31 |
| Race(white/black/Asian) | 25/8/7 | 6/9/31 |
| Diagnosis | Biopsy (n = 36) | Culture (n = 42) |
| | Radiological | Radiological |
| | (n = 4) | (n = 6) |
| Time since 1^{st} | (n = 26) | (n = 48) |
| Diagnosis <12 months | | |
| CT Stage | 1(n = 4) | |
| | 2(n = 12) | |
| | 3(n = 2) | |
| | 4(n = 2) | |
| | Unknown (n = 20) | |
| Site of Disease | | |
| Lung involvement (any) | n = 30 (69%) | n = 38 (83%) |
| Intra-thoracic | n = 6 (14%) | n = 6 (13%) |
| Lymph node (any) | | |
| Lung & Intra-thoracic | n = 17 (40%) | n = 8 (17%) |
| Lymph node (any) | | |
| Extra thoracic site (any) | n = 12 (29%) | n = 3 (7%) |

TABLE 4
Performance characteristics for ratio of CSF1R to S100A8/A9 heterodimer proteins
(measured by ELISA) in the classification of disease in the Validation cohort.

| | Cut- | | | | | | |
|---|---|---|---|---|---|---|---|
| Comparison | Off | Sensitivity | 95% CI | Specificity % | 95% CI | LR | AUC |
| All SA v All TB | 1.0 | 84.8 | 71.13% to | 83.3 | 68.64% to | 5.1 | 0.86 |
| (n = 88) | | | 93.66% | | 93.03% | | |
| SA < 1 | 1.0 | 84.8 | 71.13% to | 96.2 | 80.36% to | 22.0 | 0.93 |
| Months v All | | | 93.66% | | 99.90% | | |
| TB (n = 72) | | | | | | | |
| SA < 12 | 1.0 | 84.8 | 71.13% to | 90.3 | 74.25% to | 8.8 | 0.90 |
| Months v All | | | 93.66% | | 97.96% | | |
| TB (n = 77) | | | | | | | |
| SA > 12 | 1.0 | 84.8 | 71.13% to | 54.6 | 23.38% to | 1.9 | 0.76 |
| Months SA v | | | 93.66% | | 83.25% | | |
| All TB (57) | | | | | | | |

### Discussion

We investigated the serum proteomic signatures for patients suffering from pulmonary SA and TB. Our findings show that SA alters the expression of far fewer proteins than TB, that TB serum contains components of cellular necrosis and that two proteins CSFR1 and S100A8/A9 can provide diagnostic utility. In the SA proteomic signature ICAM1, CHIT1 and LYZ1 have all previously been found elevated in both SA and TB and we confirm this here (PMID: 18487875, PMID: 18069420, PMID: 17347558, PMID: 26270185). The protein CSF1R was highly specific to SA and to our knowledge this is the first report of CSF1R as a serum marker for SA. We demonstrate that CSF1R used in combination with S100A8/A9 provides excellent disease classification (AUC=0.86) in a large and diverse patient cohort (n=88). CSF1R is the receptor for CSF1 a key cytokine that functions in the proliferation, differentiation and survival of monocytes and macrophages (PMID:18551128). In SA alveolar macrophages that exhibit higher mitotic activity also express more CSF1R and this represents a potential source of the soluble protein found in the serum (PMID: 1694255). All proteins identified in the SA signature have direct roles in macrophage biology, are regulated by pro-inflammatory signalling (TNF-alpha, IFN-γ, LPS and ILI) and their gene mRNA's are highly expressed in monocytes and CD14+ cells (FIG. 6 B, PMID: 7905208, PMID: 892662). Taken together the serum signature observed in SA appears to be a direct result of the activity of monocytes and macrophages in disease.

Using functional annotations we compared the SA and TB proteomes and identified a TB specific increase in the abundance of proteins annotated as intracellular (cytosol, nuclear) and a common decrease in the abundance of proteins that transport lipids. In TB these functional protein groups correlated with the number of neutrophils (cytosol), the neutrophil to lymphocyte ratio (NLR) (nucleus) and total platelets (cholesterol effux) in the peripheral blood. This indicates that major changes in the TB serum proteome are at least partially dependent upon the cellular make up of patient blood. Increased neutrophil activity in TB is detectable in serum by measuring key markers (S100A8/A9, DEF1A and MPO) and detect positive correlations for these proteins with NLR (PMC4839997). In active TB loss of immune control and Mtb replication induces tissue necrosis through the lysis of infected cells driving a systemic type I interferon inflammatory response that recruits neutrophils from the lung vasculature to the site of disease (23435331). Neutrophils release azurophilic granules and genomic DNA that contain antimicrobial proteins including S100A8/A9, DEF1A and MPO (24047412). These proteins can kill Mtb and inhibit its replication but also cause further inflammation and tissue damage. The increase in abundance of intracellular proteins, dsDNA and nucleosomes in TB sera most likely originates from necrotic granulomas in the lung interstitial space (reviewed 25377142, 11244032) that drain into the peripheral blood at sites of thrombic inflammation.

We also validated the T-cell co-stimulatory cytokine ICOSLG and the adipokine RBP4 (Retinol binding protein 4), a vitamin A transport protein that links nutritional status to immune activation in TB and diabetes (PMID: 25019074, PMID: 28625041, PMID: 20032483). Both ICOSLG and RBP4 decrease in TB sera compared to both healthy and SA sera. RBP4 and ICOSLG showed no relationship with NLR or necrosis indicating that the reduction of these proteins in TB sera does not appear to reflect neutrophil activity or necrotic pathology in TB. ICOSLG did positively correlated with the abundance of blood lymphocytes in TB patients. In mice the ICOS receptor modulates the immune control of TB disease in a site-specific manner by affecting the type of T-cell response during late stage infection (PMID: 21337542, PMID: 25019074). In Chlamydia muridarum lung infection ICOSLG gene knockout leads to increased body weight loss, pathogen burden and lung pathology. The lack of ICOSLG generates an enhanced Th1 response but this is insufficient for disease control (PMID: 20190137). Our data indicates that serum ICOSLG provides a biomarker for lymphocyte abundance in TB, and it is tempting to speculate that ICOSLG is an essential factor for effective lymphocyte mediated disease containment in humans.

Using a machine leaning algorithm we used our proteomic dataset to identify a highly robust disease classification signature for SA and TB (FGA, CSF1R, ITIH1, S100A8/A9). To aid the development of this signature as a useful laboratory diagnostic test we validated our data by ELISA. Two proteins S100A8/A9 and CSF1R accurately matched our proteomic data and a simple in-patient ratio (CSF1R/S100A8/A9) performed with a similar accuracy (AUC=0.96) to the original 4 proteins. The applicability of this ratio was investigated in a unique set of SA and TB patients from the same cohort (n=88) but expanded to include TB and SA cases with more complex presentations including culture negative TB, disease at other sites outside of the lung, both within the thoracic lymphatic system at more distal sites. The ratio performed with good diagnostic performance in the cohort overall (AUC=0.86) and excellent accuracy for recently diagnosed (<1 month) pulmonary disease (AUC=0.93 sensitivity 84.8 (CI 71.1-93.7% specificity 96.2 (CI 80.4-99.9%). The test performance was affected by time since first diagnosis of SA (>12 Month AUC=0.76) driven by a decrease in serum CSF1R. This indicates that the monocyte/macrophage associated SA signature wanes as disease progresses, and may be associated with the high rate of spontaneous remissions observed in acute SA patients (PMID: 14046006, PMID: 14497750) and may also correlate with the diminished blood transcriptional host signature in inactive SA (PMID: 23940611).

In SA and TB the blood transcriptomic signature consists of a neutrophil and macrophage contained type I-interferon signature (PMID: 23940611, PMID: 22547807). This signature is generally lower in expression intensity in SA and in TB disease confined to the lymph node (PMID: 27706152) with the genes that can best differentiate TB and SA functioning in the electron transport chain, translation, cellular responses to reactive oxygen species, defence response and azurophillic granules (PMC3356621). Specific signatures have been identified able to classify TB from SA with similar sensitivity and specificity to the proteomic signature identified in our work. Bloom et al., identified a 144 gene signature able to classify TB and other diseases including SA with sensitivity (above 80%) and specificity (above 90%) validated in both independent and external cohorts (PMID:23940611). We find that sera protein profiles represent powerful measurements able to support diagnostic accuracy and as further studies are carried out similar external validation is required to prove the clinical utility indicated in our study. A limitation of our validation of CSF1R and S100A8/A9 as a diagnostic test for SA was the lack of control groups consisting of other non-TB granulomatous diseases (PMID: 25374667). These diseases (e.g. Fungi, Pneumocystis carinii, Hypersensitivity pneumonitis, chronic beryllium disease) need to be excluded when making a diagnosis for SA, they also involve macrophage activity and are often not necrotic, factors likely to be reflected in the serum and confound diagnosis using CSF1R and S100A8/A9. Despite this CSF1R and S100A8/A9 provide excellent diagnostic accuracy and the stable sensitivity of the test indicates that it would be most suitable to correctly identify as many patients with SA disease, providing a first line triage test able to find SA patients where further clinical investigations would enable other diseases to be excluded. The gold standard and only clinical marker used for SA disease activity is serum Angiotensin converting enzyme (SACE) activity. The most comprehensive investigation of SACE reported a sensitivity 58.1%, specificity 83.8% in differentiating SA from other diseases including TB (PMID: 2991971). SACE is a product of epithelioid cells that result from fused macrophages at the site of disease PMID: 3024907, and similarly to CSF1R SACE concentration in the serum returns to normal as disease progresses (PMID: 2991971). Alongside SACE, adenosine deaminase (ADA), C reactive protein (CRP), total immune globulin E (TIgE), serum amyloid A1 (SAA1) and soluble interleukin-2 receptor (sIL2R) can determine sarcoidosis activity and may be useful in diagnosis (PMID: 25623898). The activity of Adenosine ADA can separate sarcoidosis from healthy individuals with high accuracy (ROC area 0.98 CI 0.96-1.0), however ADA is also increased in TB patients as are CRP and SAA1 and thus not useful in differential diagnosis and this was confirmed in our initial proteomic screen (PMID: 25861440). sIL2R and TIgE were not detected in our study and a direct comparison with CSF1R alone or with S100A8/A9 and other necrotic factors such as nucleosomes or total serum dsDNA is required.

Through carrying out an untargeted serum proteomic screen we detected that the predominant differences between TB and SA reflect cell necrosis and neutrophil activity in TB, and macrophage and monocyte function in SA. Using two proteins that reflect this we developed and validated a simple ELISA based test. We demonstrate a high diagnostic performance in discriminating between recent TB and SA disease that appears to be more sensitive compared to transcriptomic signatures from whole blood. This test therefore has potential to be used when both these conditions are clinically indicated.

It should be understood by the skilled person that the features of the various aspects and embodiments described herein can be combined with the features of the other various aspects and embodiments.

1. A method of differentiating between sarcoidosis and tuberculosis infection in a subject, the method comprising: (a) measuring the level of a sarcoidosis-specific biomarker in a sample taken from the subject; (b) measuring the level of a tuberculosis-specific biomarker in the same sample; (c) calculating the ratio of the sarcoidosis-specific biomarker to the tuberculosis-specific biomarker; and (d) comparing the ratio of the sarcoidosis-specific biomarker to the tuberculosis-specific biomarker to one or more standard values, where a calculated ratio less than a standard value is indicative of sarcoidosis and a calculated ratio higher or equal to a standard value is indicative of tuberculosis infection.

2. The method of claim 1, wherein the sarcoidosis-specific biomarker is CSFR1 and the tuberculosis-specific biomarker is S100A8A9 (calprotectin).

3. The method of claim 3, wherein the standard value is 1.5, 2.0, 2.5, 3.0, 3.5, 4.0, 4.5 or 5.0.

4. The method of claim 1, wherein the sample is a blood sample or a serum sample.

5. The method of claim 1, wherein the measurement of the biomarker is carried out using ELISA.

6. The method of claim 1, wherein the subject is human.

7. A method of diagnosing sarcoidosis in a subject, the method comprising: (a) measuring the level of a sarcoidosis-specific biomarker in a sample taken from the subject; (b) measuring the level of a tuberculosis-specific biomarker in the same sample; (c) calculating the ratio of the sarcoidosis-specific biomarker to the tuberculosis-specific biomarker; and (d) comparing the ratio of the sarcoidosis-specific biomarker to the tuberculosis-specific biomarker to one or more standard values, where a calculated ratio less than a standard value is indicative of a positive sarcoidosis diagnosis.

8. A method of diagnosing sarcoidosis in a subject, the method comprising: (a) measuring the level of CSFR1 in a sample taken from the subject; and (d) comparing the level of CSFR1 to one or more standard values, where a CSFR1 level above a standard value is indicative of a positive sarcoidosis diagnosis.
